SEQUENCE LISTING (32 sequences)

SEQ ID NO: 1 - protein, 4 aa, synthetic construct
RRRG

SEQ ID NO: 2 - DNA, 12 bp, synthetic construct
  source 1..12: mol_type = other DNA
cgtcgccgcggt

SEQ ID NO: 3 - protein, 17 aa, synthetic construct
SGPRPRGTRGKGRRIRR

SEQ ID NO: 4 - DNA, 51 bp, synthetic construct
  source 1..51: mol_type = other DNA
tctggtcctcgtccccgtggtactcgtggtaaaggtcgccgtattcgtcgc

SEQ ID NO: 5 - protein, 19 aa, synthetic construct
SGPRPRGTRGKGRRIRRRG

SEQ ID NO: 6 - DNA, 57 bp, synthetic construct
  source 1..57: mol_type = other DNA
tctggtcctcgtccccgtggtactcgtggtaaaggtcgccgtattcgtcgccgcggt

SEQ ID NO: 7 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 8 - DNA, 498 bp, synthetic construct
  source 1..498: mol_type = other DNA
tctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaac

SEQ ID NO: 9 - protein, 1367 aa, synthetic construct
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 10 - DNA, 4101 bp, synthetic construct
  source 1..4101: mol_type = other DNA
gacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgac

SEQ ID NO: 11 - protein, 7 aa, synthetic construct
PKKKRKV

SEQ ID NO: 12 - DNA, 21 bp, synthetic construct
  source 1..21: mol_type = other DNA
ccaaagaagaagcggaaagtc

SEQ ID NO: 13 - DNA, 28 bp, synthetic construct
  source 1..28: mol_type = other DNA
ggctcgttgagctcattagctccgagcc

SEQ ID NO: 14 - DNA, 100 bp, synthetic construct
  source 1..100: mol_type = other DNA
gttttagagctagaaatagcaagttaaaataaggctagtccgttatcaacttggctcgttgagctcattagctccgagccaagtggcaccgagtcggtgc

SEQ ID NO: 15 - DNA, 100 bp, synthetic construct; identical to SEQ ID NO: 14
  source 1..100: mol_type = other DNA

SEQ ID NO: 16 - DNA, 23 bp, synthetic construct
  source 1..23: mol_type = other DNA
gtcatccagtgctaccgctgtgg

SEQ ID NO: 17 - DNA, 22 bp, synthetic construct
  source 1..22: mol_type = other DNA
gtggtagacagcatgtgtccta

SEQ ID NO: 18 - DNA, 22 bp, synthetic construct
  source 1..22: mol_type = other DNA
gtgtcaggtaatgtgctaaaca

SEQ ID NO: 19 - DNA, 22 bp, synthetic construct
  source 1..22: mol_type = other DNA
ggtggaggagggtgcatggggt

SEQ ID NO: 20 - DNA, 20 bp, synthetic construct
  source 1..20: mol_type = other DNA
tctgcttctccagccctggc

SEQ ID NO: 21 - DNA, 20 bp, synthetic construct
  source 1..20: mol_type = other DNA
gatgttccaatcagtacgca

SEQ ID NO: 22 - DNA, 18 bp, synthetic construct
  source 1..18: mol_type = other DNA
ggagtgagtacggtgtgc

SEQ ID NO: 23 - DNA, 18 bp, synthetic construct
  source 1..18: mol_type = other DNA
gagttggatgctggatgg

SEQ ID NO: 24 - DNA, 24 bp, synthetic construct
  source 1..24: mol_type = other DNA
caccgtcatccagtgctaccgctg

SEQ ID NO: 25 - DNA, 24 bp, synthetic construct
  source 1..24: mol_type = other DNA
aaaccagcggtagcactggatgac

SEQ ID NO: 26 - DNA, 43 bp, synthetic construct
  source 1..43: mol_type = other DNA
ggagtgagtacggtgtgcgtaaagaatcagggcctctggatag

SEQ ID NO: 27 - DNA, 40 bp, synthetic construct
  source 1..40: mol_type = other DNA
gagttggatgctggatggcctaccttgacaggtgaagcat

SEQ ID NO: 28 - protein, 228 aa, synthetic construct
SSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 29 - DNA, 684 bp, synthetic construct
  source 1..684: mol_type = other DNA
tcctcagagactgggcctgtcgccgtcgatccaaccctgcgccgccggattgaacctcacgagtttgaagtgttctttgacccccgggagctgagaaaggagacatgcctgctgtacgagatcaactggggaggcaggcactccatctggaggcacacctctcagaacacaaataagcacgtggaggtgaacttcatcgagaagtttaccacagagcggtacttctgccccaataccagatgtagcatcacatggtttctgagctggtccccttgcggagagtgtagcagggccatcaccgagttcctgtccagatatccacacgtgacactgtttatctacatcgccaggctgtatcaccacgcagacccaaggaataggcagggcctgcgcgatctgatcagctccggcgtgaccatccagatcatgacagagcaggagtccggctactgctggcggaacttcgtgaattattctcctagcaacgaggcccactggcctaggtacccacacctgtgggtgcgcctgtacgtgctggagctgtattgcatcatcctgggcctgcccccttgtctgaatatcctgcggagaaagcagccccagctgaccttctttacaatcgccctgcagtcttgtcactatcagaggctgccaccccacatcctgtgggccacaggcctgaag

SEQ ID NO: 30 - protein, 83 aa, synthetic construct
TNLSDIIEKETGKQLVIQESILMLPEEVEEVIGNKPESDILVHTAYDESTDENVMLLTSDAPEYKPWALVIQDSNGENKIKML

SEQ ID NO: 31 - DNA, 249 bp, synthetic construct
  source 1..249: mol_type = other DNA
actaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctg

SEQ ID NO: 32 - DNA, 22 bp, synthetic construct
  source 1..22: mol_type = other DNA
atttacagcctggcctttgggg

(19) United States
(12) Patent Application Publication
(10) Pub. No.: US 2026/0185077 A1
(43) Pub. Date: Jul. 2, 2026

(54) FUSION PROTEIN CAPABLE OF IMPROVING BASE EDITING ACTIVITY AND REDUCING OFF-TARGET LEVEL

(71) Applicant: SUZHOU INSTITUTE OF SYSTEMS MEDICINE, Suzhou (CN)

(72) Inventors: Xiaohui ZHANG, Suzhou (CN); Yinuo LI, Suzhou (CN); Yilang LIN, Suzhou (CN)

(21) Appl. No.: 19/380,923

(22) Filed: Nov. 5, 2025

(30) Foreign Application Priority Data
Nov. 5, 2024 (CN) 202411568463.8

(51) Int. Cl. C12N 15/10; C12N 9/22; C12N 9/78; C12N 15/11

(52) U.S. Cl. CPC C12N 15/102; C12N 9/226; C12N 9/78; C12N 15/111; C07K 2319/85; C12Y 305/04001; C12Y 305/04004

(57) ABSTRACT

The present disclosure provides a fusion protein capable of improving base editing activity and reducing an off-target level, and relates to the technical field of biological technologies. In the present disclosure, a degradation system based on a tDeg fusion protein is first introduced into a base editing system to obtain a novel base editing system capable of improving the base editing efficiency while reducing the off-target level. Further, a degron sequence and a RNA-binding polypeptide are fused with a base editing module of a base editor to form a fusion protein and meanwhile Pepper RNA is introduced into a loop region of sgRNA, so as to obtain the base editing system of the present application. The base editing system with high efficiency and low off-target rate constructed in the present disclosure will further promote wide base editing tool application, especially in gene therapy and clinical disease treatment.

### CROSS-REFERENCE TO RELATED APPLICATION

This application claims the priority of Chinese Patent Application No. 202411568463.8 filed with the China National Intellectual Property Administration on Nov. 5, 2024, and entitled “FUSION PROTEIN CAPABLE OF IMPROVING BASE EDITING ACTIVITY AND REDUCING OFF-TARGET LEVEL”, the disclosure of which is incorporated by reference herein in its entirety as part of the present application.

### SEQUENCE LISTING

The present specification refers to a Sequence Listing, which is submitted electronically as a.xml file named “JUNHO003US_Sequence_Listing.xml”. The.xml file was generated on Nov. 5, 2025 and is 36,700 bytes in size. The entire contents of the Sequence Listing are herein incorporated by reference

### TECHNICAL FIELD

The present application relates to the technical field of biological technologies, particularly to a fusion protein capable of improving base editing activity and reducing an off-target level.

### BACKGROUND

Studies have shown that approximately 60% of hereditary diseases are caused by single-base mutations. The restorative treatment of these hereditary diseases is completed by utilizing traditional CRISPR-mediated homologous recombination, with extremely low efficiency and poor therapeutic effect. A new-generation gene editing technology, i.e., a base editing technology, can achieve efficient base conversion, thereby bringing a hope for curing hereditary diseases caused by single-base mutations. At present, the base editing technology is mainly represented by cytosine base editing (CBE) and adenine base editing (ABE) in which cytosine deaminase or adenine deaminase is fused with Cas9. CBE and ABE can respectively achieve efficient C-to-T and A-to-G base conversions at specific sites of a genome, and are suitable for treatment and correction of 16% and 47% hereditary diseases caused by base mutations respectively.

Base editors initially had relatively low editing efficiency. After years of development, their editing efficiency has been effectively improved through approaches such as directed evolution and rational design, leading to generation of relatively efficient versions such as ABE8e, ABE8r, BE4max and A3A-BE3. However, the improvement of the editing efficiency of base editors in these efficient versions is limited, which also brings new security risks, such as an unpredictable DNA off-targeting in a genome.

To sum up, currently, there is an urgent need for a base editing tool that balances editing accuracy and high editing efficiency.

### SUMMARY

To further improve the editing efficiency while reducing the off-target risk, in the present disclosure, a degradation system based on a tDeg fusion protein is introduced into a base editing system, thereby establishing a new strategy for improving the editing efficiency while reducing the off-target level.

On the one hand, the present application provides a fusion protein, the fusion protein including a protein degradation module (tDeg) and a base editing module, wherein the protein degradation module includes a degron and an RNA-binding polypeptide (Tat peptide).

Further, the protein degradation module is located at the C-terminus of the fusion protein; optionally, the RNA-binding polypeptide and the degron are connected in sequence from the N-terminus to the C-terminus of a sequence.

Two amino acids in the degron are shared with the RNA-binding polypeptide, and therefore their position order (from the N-terminus to the C-terminus of the sequence) is as follows: RNA-binding polypeptide-degron.

Further, the degron includes an amino acid sequence as shown in SEQ ID NO. 1 or an amino acid sequence having at least 98% sequence identity to SEQ ID NO. 1.

The degron includes the amino acid sequence as shown in SEQ ID NO. 1 or the amino acid sequence having at least 98%, 99%, 99.1%, 99.2%, 99.3%, 99.4%, 99.5%, 99.6%, 99.7%, 99.8% or 99.9% sequence identity to SEQ ID NO. 1.

Optionally, the sequence encoding the degron includes a nucleotide sequence as shown in SEQ ID NO. 2 or a nucleotide sequence having at least 98%, 99%, 99.1%, 99.2%, 99.3%, 99.4%, 99.5%, 99.6%, 99.7%, 99.8% or 99.9% sequence identity to SEQ ID NO. 2.

Further, the RNA-binding polypeptide includes an amino acid sequence as shown in SEQ ID NO. 3 or an amino acid sequence having at least 98% sequence identity to SEQ ID NO. 3.

Optionally, the RNA-binding polypeptide includes the amino acid sequence as shown in SEQ ID NO. 3 or the amino acid sequence having at least 98%, 99%, 99.1%, 99.2%, 99.3%, 99.4%, 99.5%, 99.6%, 99.7%, 99.8% or 99.9% sequence identity to SEQ ID NO. 3.

Optionally, the sequence encoding the RNA-binding polypeptide includes a nucleotide sequence as shown in SEQ ID NO. 4 or a nucleotide sequence having at least 98%, 99%, 99.1%, 99.2%, 99.3%, 99.4%, 99.5%, 99.6%, 99.7%, 99.8% or 99.9% sequence identity to SEQ ID NO. 4.

More optionally, the protein degradation module includes an amino acid sequence as shown in SEQ ID NO. 5 or an amino acid sequence having at least 98%, 99%, 99.1%, 99.2%, 99.3%, 99.4%, 99.5%, 99.6%, 99.7%, 99.8% or 99.9% sequence identity to SEQ ID NO. 5.

The sequence encoding the protein degradation module includes a nucleotide sequence as shown in SEQ ID NO. 6 or a nucleotide sequence having at least 98%, 99%, 99.1%, 99.2%, 99.3%, 99.4%, 99.5%, 99.6%, 99.7%, 99.8% or 99.9% sequence identity to SEQ ID NO. 6.

Further, the base editing module includes glycosylase, nucleoside deaminase and/or nuclease.

The base editing module can be known functional modules that are capable of achieving base editing in a base editor, or a combination thereof. The base editor includes, but not limited to, one or more of base editors at the DNA level, such as ABE, CBE, ACBE, CGBE, AYBE and gGBE, or base editors at the RNA level, such as REPAIR and RESCUE.

In an optional embodiment, the protein degradation module (tDeg) is introduced for modification by taking an ABE base editing system as an example.

In an optional embodiment, the protein degradation module (tDeg) is introduced for modification by taking a CBE base editing system as an example.

Further, the nucleoside deaminase includes cytosine deaminase and/or adenosine deaminase; optionally, the nucleoside deaminase is adenosine deaminase; more optionally, the adenosine deaminase is TadA-8e; more optionally, the amino acid sequence of the adenosine deaminase includes a sequence as shown in SEQ ID NO. 7 or an amino acid sequence having at least 98% sequence identity to SEQ ID NO. 7; more optionally, a nucleotide sequence encoding the adenosine deaminase includes a sequence as shown in SEQ ID NO. 8 or a nucleotide sequence having at least 98% sequence identity to SEQ ID NO. 8.

Optionally, the nucleoside deaminase is cytosine deaminase; more optionally, the cytosine deaminase is rAPOBEC; more optionally, the amino acid sequence of the cytosine deaminase includes a sequence as shown in SEQ ID NO. 28 or an amino acid sequence having at least 98% sequence identity to SEQ ID NO. 28; more optionally, a nucleotide sequence encoding the cytosine deaminase comprises a sequence as shown in SEQ ID NO. 29 or a nucleotide sequence having at least 98% sequence identity to SEQ ID NO. 29.

Further, the nuclease is selected from one or more of Cas9, Cas3, Cas8a, Cas8b, Cas10d, Cse1, Csy1, Csn2, Cas4, Cas10, Csm2, Cmr5, Fok1 and Cpf1; optionally, the nuclease is Cas9; more optionally, the Cas9 is nCas9; more optionally, the Cas9 is spCas9n; more optionally, the amino acid sequence of the spCas9n includes an amino acid sequence as shown in SEQ ID NO. 9 or an amino acid sequence having at least 98% sequence identity to SEQ ID NO. 9; more optionally, the encoding sequence of the spCas9n includes a nucleotide sequence as shown in SEQ ID NO. 10 or a nucleotide sequence having at least 98% sequence identity to SEQ ID NO. 10.

Further, the fusion protein further includes a nuclear localization signal (NLS); optionally, the NLS is located at least one end of the fusion protein; more optionally, the NLS is located at both ends of the fusion protein.

It will be understood by those skilled in the art that the fusion protein of the present application can be constructed by using the known NLS sequence.

In an optional embodiment, the amino acid sequence of the NLS is as shown in SEQ ID NO. 11; and the encoding sequence of the NLS is as shown in SEQ ID NO. 12.

Further, the fusion protein further includes at least one uracil glycosylase inhibitor (UGI); optionally, the fusion protein has two UGIs.

In an optional embodiment, the amino acid sequence of the UGI is as shown in SEQ ID NO. 30; and the encoding sequence of the UGI is as shown in SEQ ID NO. 31.

It will be understood by those skilled in the art that a known Linker can be added between the protein degradation module and the base editing module for linkage, and on the premise that the function of the fusion protein itself is not affected, the fusion protein can also be subjected to known modifications, including phosphorylation, acetylation, ubiquitination, glycosylation, etc.

In an optional embodiment, the base editing module and the protein degradation module in the fusion protein are successively linked from the N-terminus to the C-terminus of the sequence.

In an optional embodiment, the NLS, the adenine deaminase, the nuclease, the NLS, and the protein degradation module in the fusion protein are successively linked from the N-terminus to the C-terminus of the sequence.

In an optional embodiment, the NLS, the cytosine deaminase, the nuclease, the UGI, the UGI, the NLS and the protein degradation module in the fusion protein are successively linked from the N-terminus to the C-terminus of the sequence.

In another aspect, the present application further provides a biological material, the biological material including any one of the following A)-D):
A) a gene encoding the fusion protein;
B) an expression cassette containing the gene in A);
C) a recombinant vector containing the gene in A) and/or the expression cassette in B); and
D) a recombinant cell or recombinant bacterium containing the fusion protein, the gene in A), the expression cassette in B) and/or the recombinant vector in C).

The expression cassette described herein may further include functional elements such as promoters, terminators and marker genes. Those skilled in the art can make routine selection to the above functional elements according to actual situations, as long as they can complete the expression of the gene in A). There is no further restriction on the structure and composition of the expression cassette here.

The vector described herein refers to a vector that is capable of transporting foreign DNA or target genes into host cells for amplification and expression. The vector can be a cloning vector or an expression vector. Those skilled in the art can make selection to the above vectors according to actual situations, and there is no further restriction.

It should be understood that those skilled in the art can select a proper gene editing system and gene editing method according to actual situations to complete the modification of the above mutants.

In another aspect, the present application further provides a base editing system, the base editing system including the fusion protein or the biological material.

Optionally, the base editing system is a single-base gene editing system.

Further, the system further includes sgRNA. The sgRNA guides the fusion protein to perform base editing on a target gene; optionally, the sgRNA further includes a RNA-binding polypeptide response sequence (TAR, Pepper RNA) which binds to the RNA-binding polypeptide; more optionally, the RNA-binding polypeptide response sequence includes a nucleotide sequence as shown in SEQ ID NO. 13 or a nucleotide sequence having at least 98% sequence identity to SEQ ID NO. 13.

More optionally, the RNA-binding polypeptide response sequence includes the nucleotide sequence as shown in SEQ ID NO. 13 or the nucleotide sequence having at least 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8% or 99.9% sequence identity to SEQ ID NO. 13.

In an optional embodiment, an editing target is ABLIM3, and the sequence of human endogenous target ABLIM3 is as shown in SEQ ID NO. 16.

Optionally, the RNA-binding polypeptide is inserted into the loop region of the sgRNA. The sequence of the sgRNA is as shown in SEQ ID NO. 14.

In an optional embodiment, the sequence of the RNA-binding polypeptide after being inserted into the sgRNA is as shown in SEQ ID NO. 15.

It will be understood by those skilled in the art that in the present application, only the target ABLIM3 is taken as an example, on that basis, other editing targets can be selected and designed for gene editing operation. There is no further restriction here.

In an optional embodiment, when the base editing module is ABE, the system can achieve A-to-G single-base editing.

In an optional embodiment, when the base editing module is CBE, the system can achieve C-to-T single-base editing.

In another aspect, the present application further provides use of a protein degradation module in a base editing system, wherein the protein degradation module includes a degron and an RNA-binding polypeptide; optionally, the use includes reducing the off-target level of the base editing system and/or improving the gene editing efficiency of the base editing system.

The base editing system includes, but not limited to, one or more of base editing systems at the DNA level, such as ABE, CBE, ACBE, CGBE, AYBE and gGBE, or base editing systems at the RNA level, such as REPAIR and RESCUE.

In another aspect, the present application further provides use of the fusion protein, or the biological material, or the base editing system in reducing the off-target level and/or improving the gene editing efficiency.

Further, the off-target level includes off-targeting at the DNA level and/or off-targeting at the RNA level; optionally, the off-targeting at the DNA level include sgRNA-dependent off-targeting and/or non-Cas9-dependent off-targeting.

In the present application, the testing of the off-target level is performed by using a non-Cas9-dependent off-target level as an example.

In another aspect, the present application further provides use of the fusion protein, or the biological material, or the base editing system in gene editing for the purposes of non-disease diagnosis and therapy and/or preparation of gene editing products.

The present disclosure has the following benefits:

In the present application, the degradation system based on the tDeg fusion protein is first introduced into the base editing system to obtain a novel base editing system capable of improving the base editing efficiency while reducing the off-target level. Further, the degron sequence and the RNA-binding polypeptide are fused with the base editing module of the base editor to obtain the fusion protein and meanwhile Pepper RNA is introduced into the loop region of the sgRNA, so as to obtain the base editing system of the present application.

The base editing system with high efficiency and a low off-target rate constructed in the present application will further promote the wide application of the base editing tool, especially in gene therapy and clinical disease treatment.

### BRIEF DESCRIPTION OF THE DRAWINGS

The accompanying drawings described here are used for providing further understanding of the present application, and constitute a part of the present application. The exemplary embodiments and their descriptions are used for explaining but not properly limiting the present application. In the drawings:

FIG. 1 is a working principle diagram of ABE8e++;

FIG. 2 is a working principle diagram of BE4max++;

FIG. 3 is a construction schematic diagram of ABE8e++;

FIG. 4 is a construction schematic diagram of BE4max++;

FIG. 5 is an editing efficiency comparison diagram of ABE8e and ABE8e++ at a human endogenous target ABLIM3;

FIG. 6 is an off-targeting comparison diagram of ABE8e and ABE8e++;

FIG. 7 is an editing efficiency comparison diagram of BE4max and BE4max++ at a human endogenous target ABLIM3; and

FIG. 8 is an off-targeting comparison diagram of BE4max and BE4max++.

### DETAILED DESCRIPTION OF THE EMBODIMENTS

To more clearly demonstrate the overall concept of the present application, the present application will be described in detail with reference to accompanying drawings by way of embodiments. In the following description, a lot of specific details are given so as to provide more thorough understanding for the present disclosure. However, it is obvious for those skilled in the art that the present disclosure is not implemented without one or more of these details. In other examples, some technical features known in the art are not described in order to avoid confusing with the present disclosure.

It should be noted that the following detailed description is exemplary, aiming at providing further description of the present application. Unless otherwise stated, all technical and scientific terms used herein have the same meaning as those generally understood that persons of ordinary skill in the art to which the present application pertains.

It is noted that the terms used herein are merely for describing specific embodiments and are not intended to limit the exemplary embodiments according to the present application. As used herein, singular forms are also intended to include plural forms, unless the context clearly indicates otherwise. In addition, it should be understood that when the terms “comprise” and/or “include” are used in this specification, they specify the presence of features, steps, operations, devices, components and/or combinations thereof.

Unless otherwise specified, in the following embodiments, all reagents or instruments whose manufacturers are not indicated are conventional products commercially available. The products plasmids, restriction enzymes, PCR enzymes, column-based DNA extraction kits and DNA gel recovery kits used in the following examples are commercial products, and specific operations are performed in accordance with the kit instructions.

Before further describing the specific embodiments of the present disclosure, it should be understood that the protective scope of the present disclosure is not limited to the specific embodiments described below; it should also be understood that the terms used in the embodiments of the present disclosure are for describing specific embodiments but not for limiting the protective scope of the present disclosure. For test methods without specified specific conditions in the following examples, conventional conditions or conditions recommended by respective manufacturers are usually adopted.

When the examples provide numerical ranges, it should be understood that unless otherwise states otherwise, both endpoints of each numerical value range and any value between the two endpoints can be selected. Unless otherwise defined, all the technical and scientific terms used in the present disclosure have the same meanings as those commonly understood by those skilled in the art. In addition to the specific methods, equipment and materials used in embodiments, any methods, equipment and materials of the existing technology that are similar or equivalent to those described in the embodiments of the present disclosure can also be used to implement the present disclosure, based on the existing technology mastered by those skilled in the art and the records of the present disclosure.

Unless otherwise stated, the experimental methods, detection methods and preparation methods disclosed in the present disclosure all adopt conventional techniques in the fields of molecular biology, biochemistry, chromatin structure and analysis, analytical chemistry, cell culture, a recombinant DNA technology and related fields that are conventional in the art. Specifically, they can be performed with reference to Molecular Cloning: A Laboratory Manual (Fourth Edition).

In this specification, the amino acids at the corresponding positions are represented by the recognized IUPAC single-letter abbreviations, where each amino acid and its abbreviation are as follows: alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

### 1.1 Design and Construction of Plasmids

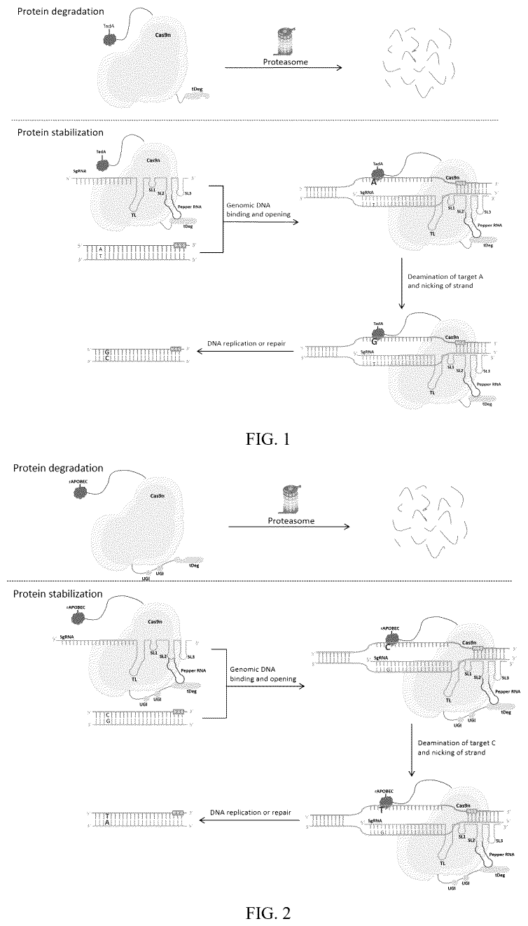

1.1.1 In the present disclosure, a degron sequence (Arg-Arg-Arg-Gly) and an RNA-binding peptide (Tat peptide) were introduced into ABE8e (wherein, the degron sequence and the RNA-binding peptide constituted tDeg), and meanwhile Pepper RNA was introduced into the loop region of sgRNA. When a fusion protein existed independently, the degron recruited proteasomes to degrade the fusion protein; when the fusion protein bound to the corresponding sgRNA, the Tat peptide bound to TAR (Pepper RNA) to protect the degron, thereby preventing the recruitment of the proteasomes required for protein hydrolysis and enhancing the stability of the fusion protein (as shown in FIG. 1). An accurate adenine base editor ABE8e++ capable of improving the base editing activity while reducing the off-target level was developed.

By using the same method, the degron and the RNA-binding peptide were introduced into BE4max, and meanwhile Pepper RNA was introduced into the loop region of the sgRNA. An accurate cytosine base editor BE4max++ capable of improving the base editing activity while reducing the off-target level was developed, as shown in FIG. 2.

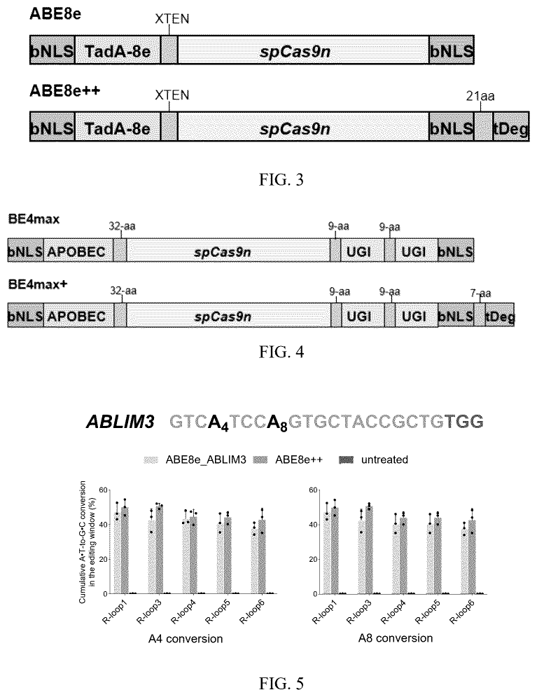

In the present disclosure, the fusion protein was constructed by using a ClonExpress MultiS one-step cloning kit (Vazyme) based on an ABE8e main chain. ABE8e (#138489) plasmids were obtained from Addgene. The polymerase chain reaction (PCR) was performed using a KOD-Plus-Neo DNA polymerase (Toyobo, Cat. No.: KOD-401). The structural diagram of the fusion protein ABE8e++ is as shown in FIG. 3, and the related sequences are listed in Table 1. BE4max++ was constructed by using the same method. BE4max (#112096) plasmids were obtained from Addgene. The structural diagram of the fusion protein BE4max++ is as shown in FIG. 4, and the related sequences are listed in Table 1.

TABLE 1
Proteins and sequences

| Names | Base sequence(5′-3′) | Amino acid sequence |
|---|---|---|
| TadA-8e | tctgaggtggagttttcccacgagtactggatgagacatgccctgac | SEVEFSHEYWMRHALT |
| | cctggccaagagggcacgggatgagagggaggtgcctgtgggag | LAKRARDEREVPVGAV |
| | ccgtgctggtgctgaacaatagagtgatcggcgagggctggaaca | LVLNNRVIGEGWNRAI |
| | gagccatcggcctgcacgacccaacagcccatgccgaaattatggc | GLHDPTAHAEIMALRQ |
| | cctgagacagggcggcctggtcatgcagaactacagactgattgac | GGLVMQNYRLIDATLY |
| | gccaccctgtacgtgacattcgagccttgcgtgatgtgcgccggcgc | VTFEPCVMCAGAMIHS |
| | catgatccactctaggatcggccgcgtggtgtttggcgtgaggaact | RIGRVVFGVRNSKRGA |
| | caaaaagaggcgccgcaggctccctgatgaacgtgctgaactacc | AGSLMNVLNYPGMNH |
| | ccggcatgaatcaccgcgtcgaaattaccgagggaatcctggcaga | RVEITEGILADECAALL |
| | tgaatgtgccgccctgctgtgcgatttctatcggatgcctagacaggt | CDFYRMPRQVFNAQK |
| | gttcaatgctcagaagaaggcccagagctccatcaac (SEQ ID | KAQSSIN (SEQ ID |
| | NO. 8) | NO. 7) |
| spCas9n | gacaagaagtacagcatcggcctggccatcggcaccaactctgtgg | DKKYSIGLAIGTNSVG |
| | gctgggccgtgatcaccgacgagtacaaggtgcccagcaagaaatt | WAVITDEYKVPSKKFK |
| | caaggtgctgggcaacaccgaccggcacagcatcaagaagaacct | VLGNTDRHSIKKNLIG |
| | gatcggagccctgctgttcgacagcggcgaaacagccgaggccac | ALLFDSGETAEATRLKR |
| | ccggctgaagagaaccgccagaagaagatacaccagacggaaga | TARRRYTRRKNRICYL |
| | accggatctgctatctgcaagagatcttcagcaacgagatggccaag | QEIFSNEMAKVDDSFF |
| | gtggacgacagcttcttccacagactggaagagtccttcctggtgga | HRLEESFLVEEDKKHE |
| | agaggataagaagcacgagcggcaccccatcttcggcaacatcgt | RHPIFGNIVDEVAYHEK |
| | ggacgaggtggcctaccacgagaagtaccccaccattaccacctg | YPTIYHLRKKLVDSTD |
| | agaaagaaactggtggacagcaccgacaaggccgacctgcggct | KADLRLIYLALAHMIK |
| | gatctatctggccctggcccacatgatcaagttccggggccacttcct | FRGHFLIEGDLNPDNSD |
| | gatcgagggcgacctgaaccccgacaacagcgacgtggacaagc | VDKLFIQLVQTYNQLF |
| | tgttcatccagctggtgcagacctacaaccagctgttcgaggaaaac | EENPINASGVDAKAILS |
| | cccatcaacgccagcggcgtggacgccaaggccatcctgtctgcc | ARLSKSRRLENLIAQLP |
| | agactgagcaagagcagacggctggaaaatctgatcgcccagctg | GEKKNGLFGNLIALSL |
| | cccggcgagaagaagaatggcctgttcggaaacctgattgccctga | GLTPNFKSNFDLAEDA |
| | gcctgggcctgacccccaacttcaagagcaacttcgacctggccga | KLQLSKDTYDDDLDNL |
| | ggatgccaaactgcagctgagcaaggacacctacgacgacgacct | LAQIGDQYADLFLAAK |
| | ggacaacctgctggcccagatcggcgaccagtacgccgacctgttt | NLSDAILLSDILRVNTEI |
| | ctggccgccaagaacctgtccgacgccatcctgctgagcgacatcc | TKAPLSASMIKRYDEH |
| | tgagagtgaacaccgagatcaccaaggcccccctgagcgcctctat | HQDLTLLKALVRQQLP |
| | gatcaagagatacgacgagcaccaccaggacctgaccctgctgaa | EKYKEIFFDQSKNGYA |
| | agctctcgtgcggcagcagctgcctgagaagtacaaagagattttctt | GYIDGGASQEEFYKFIK |
| | cgaccagagcaagaacggctacgccggctacattgacggcggag | PILEKMDGTEELLVKL |
| | ccagccaggaagagttctacaagttcatcaagcccatcctggaaaa | NREDLLRKQRTFDNGSI |
| | gatggacggcaccgaggaactgctcgtgaagctgaacagagagg | PHQIHLGELHAILRRQE |
| | acctgctgcggaagcagcggaccttcgacaacggcagcatccccc | DFYPFLKDNREKIEKIL |
| | accagatccacctgggagagctgcacgccattctgcggcggcagg | TFRIPYYVGPLARGNSR |
| | aagatttttacccattcctgaaggacaaccgggaaaagatcgagaag | FAWMTRKSEETITPWN |
| | atcctgaccttccgcatcccctactacgtgggccctctggccagggg | FEEVVDKGASAQSFIER |
| | aaacagcagattcgcctggatgaccagaaagagcgaggaaaccat | MTNFDKNLPNEKVLPK |
| | caccccctggaacttcgaggaagtggtggacaagggcgcttccgc | HSLLYEYFTVYNELTK |
| | ccagagcttcatcgagcggatgaccaacttcgataagaacctgccc | VKYVTEGMRKPAFLSG |
| | aacgagaaggtgctgcccaagcacagcctgctgtacgagtacttca | EQKKAIVDLLFKTNRK |
| | ccgtgtataacgagctgaccaaagtgaaatacgtgaccgagggaat | VTVKQLKEDYFKKIEC |
| | gagaaagcccgccttcctgagcggcgagcagaaaaaggccatcgt | FDSVEISGVEDRFNASL |
| | ggacctgctgttcaagaccaaccggaaagtgaccgtgaagcagctg | GTYHDLLKIIKDKDFLD |
| | aaagaggactacttcaagaaaatcgagtgcttcgactccgtggaaat | NEENEDILEDIVLTLTLF |
| | ctccggcgtggaagatcggttcaacgcctccctgggcacataccac | EDREMIEERLKTYAHL |
| | gatctgctgaaaattatcaaggacaaggacttcctggacaatgagga | FDDKVMKQLKRRRYT |
| | aaacgaggacattctggaagatatcgtgctgaccctgacactgtttga | GWGRLSRKLINGIRDK |
| | ggacagagagatgatcgaggaacggctgaaaacctatgcccacct | QSGKTILDFLKSDGFAN |
| | gttcgacgacaaagtgatgaagcagctgaagcggcggagatacac | RNFMQLIHDDSLTFKE |
| | cggctggggcaggctgagccggaagctgatcaacggcatccggg | DIQKAQVSGQGDSLHE |
| | acaagcagtccggcaagacaatcctggatttcctgaagtccgacgg | HIANLAGSPAIKKGILQ |
| | cttcgccaacagaaacttcatgcagctgatccacgacgacagcctga | TVKVVDELVKVMGRH |
| | cctttaaagaggacatccagaaagcccaggtgtccggccagggcg | KPENIVIEMARENQTT |
| | atagcctgcacgagcacattgccaatctggccggcagccccgccat | QKGQKNSRERMKRIEE |
| | taagaagggcatcctgcagacagtgaaggtggtggacgagctcgt | GIKELGSQILKEHPVEN |
| | gaaagtgatgggccggcacaagcccgagaacatcgtgatcgaaat | TQLQNEKLYLYYLQNG |
| | ggccagagagaaccagaccacccagaagggacagaagaacagc | RDMYVDQELDINRLSD |
| | cgcgagagaatgaagcggatcgaagagggcatcaaagagctggg | YDVDHIVPQSFLKDDSI |
| | cagccagatcctgaaagaacaccccgtggaaaacacccagctgca | DNKVLTRSDKNRGKSD |
| | gaacgagaagctgtacctgtactacctgcagaatgggcgggatatgt | NVPSEEVVKKMKNYW |
| | acgtggaccaggaactggacatcaaccggctgtccgactacgatgt | RQLLNAKLITQRKFDN |
| | ggaccatatcgtgcctcagagctttctgaaggacgactccatcgaca | LTKAERGGLSELDKAG |
| | acaaggtgctgaccagaagcgacaagaaccggggcaagagcgac | FIKRQLVETRQITKHVA |
| | aacgtgccctccgaagaggtcgtgaagaagatgaagaactactggc | QILDSRMNTKYDENDK |
| | ggcagctgctgaacgccaagctgattacccagagaaagttcgacaa | LIREVKVITLKSKLVSD |
| | tctgaccaaggccgagagaggcggcctgagcgaactggataagg | FRKDFQFYKVREINNY |
| | ccggcttcatcaagagacagctggtggaaacccggcagatcacaa | HHAHDAYLNAVVGTA |
| | agcacgtggcacagatcctggactcccggatgaacactaagtacga | LIKKYPKLESEFVYGD |
| | cgagaatgacaagctgatccgggaagtgaaagtgatcaccctgaag | YKVYDVRKMIAKSEQ |
| | tccaagctggtgtccgatttccggaaggatttccagttttacaaagtgc | EIGKATAKYFFYSNIMN |
| | gcgagatcaacaactaccaccacgcccacgacgcctacctgaacg | FFKTEITLANGEIRKRP |
| | ccgtcgtgggaaccgccctgatcaaaaagtaccctaagctggaaag | LIETNGETGEIVWDKG |
| | cgagttcgtgtacggcgactacaaggtgtacgacgtgcggaagatg | RDFATVRKVLSMPQVN |
| | atcgccaagagcgagcaggaaatcggcaaggctaccgccaagtac | IVKKTEVQTGGFSKESI |
| | ttcttctacagcaacatcatgaactttttcaagaccgagattaccctgg | LPKRNSDKLIARKKDW |
| | ccaacggcgagatccggaagcggcctctgatcgagacaaacggc | DPKKYGGFDSPTVAYS |
| | gaaaccggggagatcgtgtgggataagggccgggattttgccacc | VLVVAKVEKGKSKKLK |
| | gtgcggaaagtgctgagcatgccccaagtgaatatcgtgaaaaaga | SVKELLGITIMERSSFE |
| | ccgaggtgcagacaggcggcttcagcaaagagtctatcctgcccaa | KNPIDFLEAKGYKEVK |
| | gaggaacagcgataagctgatcgccagaaagaaggactgggacc | KDLIIKLPKYSLFELEN |
| | ctaagaagtacggcggcttcgacagccccaccgtggcctattctgtg | GRKRMLASAGELQKG |
| | ctggtggtggccaaagtggaaaagggcaagtccaagaaactgaag | NELALPSKYVNFLYLA |
| | agtgtgaaagagctgctggggatcaccatcatggaaagaagcagct | SHYEKLKGSPEDNEQK |
| | tcgagaagaatcccatcgactttctggaagccaagggctacaaaga | QLFVEQHKHYLDEIIEQ |
| | agtgaaaaaggacctgatcatcaagctgcctaagtactccctgttcga | ISEFSKRVILADANLDK |
| | gctggaaaacggccggaagagaatgctggcctctgccggcgaact | VLSAYNKHRDKPIREQ |
| | gcagaagggaaacgaactggccctgccctccaaatatgtgaacttc | AENIIHLFTLTNLGAPA |
| | ctgtacctggccagccactatgagaagctgaagggctcccccgagg | AFKYFDTTIDRKRYTST |
| | ataatgagcagaaacagctgtttgtggaacagcacaagcactacctg | KEVLDATLIHQSITGLY |
| | gacgagatcatcgagcagatcagcgagttctccaagagagtgatcc | ETRIDLSQLGGD (SEQ |
| | tggccgacgctaatctggacaaagtgctgtccgcctacaacaagca | ID NO. 9) |
| | ccgggataagcccatcagagagcaggccgagaatatcatccacct | |
| | gtttaccctgaccaatctgggagcccctgccgccttcaagtactttga | |
| | caccaccatcgaccggaagaggtacaccagcaccaaagaggtgct | |
| | ggacgccaccctgatccaccagagcatcaccggcctgtacgagac | |
| | acggatcgacctgtctcagctgggaggtgac (SEQ ID NO. | |
| | 10) | |
| Tat | tctggtcctcgtccccgtggtactcgtggtaaaggtcgccgtattcgtc | SGPRPRGTRGKGRRIR |
| peptide | gc (SEQ ID NO. 4) | R (SEQ ID NO. 3) |
| degron | cgtcgccgcggt (SEQ ID NO. 2) | RRRG (SEQ ID NO. 1) |
| tDeg | tctggtcctcgtccccgtggtactcgtggtaaaggtcgccgtattcgtc | SGPRPRGTRGKGRRIR |
| | gccgcggt (SEQ ID NO. 6) | RRG (SEQ ID NO. 5) |
| NLS | ccaaagaagaagcggaaagtc (SEQ ID NO. 12) | PKKKRKV |
| | | (SEQ ID NO. 11) |
| rAPOBEC | tcctcagagactgggcctgtcgccgtcgatccaaccctgcgccgcc | SSETGPVAVDPTLRRRI |
| | ggattgaacctcacgagtttgaagtgttctttgacccccgggagctga | EPHEFEVFFDPRELRKE |
| | gaaaggagacatgcctgctgtacgagatcaactggggaggcaggc | TCLLYEINWGGRHSIW |
| | actccatctggaggcacacctctcagaacacaaataagcacgtgga | RHTSQNTNKHVEVNFI |
| | ggtgaacttcatcgagaagtttaccacagagcggtacttctgcccca | EKFTTERYFCPNTRCSI |
| | ataccagatgtagcatcacatggtttctgagctggtccccttgcggag | TWFLSWSPCGECSRAI |
| | agtgtagcagggccatcaccgagttcctgtccagatatccacacgtg | TEFLSRYPHVTLFIYIAR |
| | acactgtttatctacatcgccaggctgtatcaccacgcagacccaag | LYHHADPRNRQGLRDL |
| | gaataggcagggcctgcgcgatctgatcagctccggcgtgaccatc | ISSGVTIQIMTEQESGY |
| | cagatcatgacagagcaggagtccggctactgctggcggaacttcg | CWRNFVNYSPSNEAH |
| | tgaattattctcctagcaacgaggcccactggcctaggtacccacac | WPRYPHLWVRLYVLEL |
| | ctgtgggtgcgcctgtacgtgctggagctgtattgcatcatcctgggc | YCIILGLPPCLNILRRKQ |
| | ctgcccccttgtctgaatatcctgcggagaaagcagccccagctgac | PQLTFFTIALQSCHYQR |
| | cttctttacaatcgccctgcagtcttgtcactatcagaggctgccaccc | LPPHILWATGLK (SEQ |
| | cacatcctgtgggccacaggcctgaag (SEQ ID NO. 29) | ID NO. 28) |
| UGI | actaatctgagcgacatcattgagaaggagactgggaaacagctgg | TNLSDIIEKETGKQLVI |
| | tcattcaggagtccatcctgatgctgcctgaggaggtggaggaagtg | QESILMLPEEVEEVIGN |
| | atcggcaacaagccagagtctgacatcctggtgcacaccgcctacg | KPESDILVHTAYDESTD |
| | acgagtccacagatgagaatgtgatgctgctgacctctgacgccccc | ENVMLLTSDAPEYKPW |
| | gagtataagccttgggccctggtcatccaggattctaacggcgagaa | ALVIQDSNGENKIKML |
| | taagatcaagatgctg (SEQ ID NO. 31) | (SEQ ID NO. 30) |

In the present disclosure, a trans-activation response element (TAR), Pepper RNA, corresponding to the RNA-binding peptide (Tat peptide) was fused into the loop region of the sgRNA, and a sgRNA vector fused with Pepper RNA was synthesized by Genewiz (Suzhou, China). Further, the Pepper RNA (ggctcgttgagctcattagctccgagcc, SEQ ID NO. 13) sequence was inserted into the loop region of the sgRNA (the sequence was gttttagagctagaaatagcaagttaaaataaggctagtccgttatcaacttaagtggcaccgagtcggtgc, SEQ ID NO. 14) to obtain the sgRNA vector (the sequence was gttttagagctagaaatagcaagttaaaataaggctagtccgttatcaacttggctcgttgagctcattagctccgagccaagtggcaccgagt cggtgc, SEQ ID NO.15) fused with Pepper RNA. For the construction of the sgRNA expression plasmids, oligonucleotides (Oligo in Table 2) were annealed for 5 min at 95° C., then cooled to room temperature, and linked to a synthesized BbsI linearized vector (Thermo Fisher Scientific) of the sgRNA containing Pepper RNA, so as to obtain ABLIM3-Pepper RNA.

In the present disclosure, the targeted editing efficiency of ABE8e++ or BE4max++ was tested by taking the human endogenous target ABLIM3 (GTCATCCAGTGCTACCGCTGTGG, SEQ ID NO. 16) as an example; the unpredictable DNA off-targeting was evaluated by utilizing an improved R-loop experiment, namely, Cas9-non-dependent DNA off-target analysis was performed by replacing dSaCas9-sgRNA plasmids with nSaCas9-sgRNA plasmids at the orthogonal R-loop site in the genome (reference: Doman, J. L., Raguram, A., Newby, G. A. et al. Evaluation and minimization of Cas9-independent off-target DNA editing by cytosine base editors. Nat Biotechnol 38, 620-628 (2020). https://doi.org/10.1038/s41587-020-0414-6). The nSaCas9-sgRNA (#138162) plasmids were obtained from Addgene.

1.1.2 The plasmids constructed in 1.1.1 and related sequences were sequenced via Sanger to ensure complete correction.

### 1.2 Cell Transfection

HEK293T 2×10⁵ cells were seeded on a 24-well plate. When the cells reached 70%-80% confluency, a composite comprising 3 μL of polyethyleneimine (PEI, Polysciences), 1 μg of plasmid DNA (375 ng of nSaCas9-sgRNA plasmids, 375 ng of base editing plasmids and 250 ng of sgRNA plasmids) was added into the cells, and 3 replicate wells were set for combined transfection of each plasmid, with 2×10⁵ cells per well. Meanwhile, a blank control without any transfected plasmids was set. After transfection for 24 h and 48 h respectively, supernatant was discarded, 500 μL of complete culture medium (DMEM+FBS+PS) was added; and after 72 h, the cells were digested with 0.25% trypsin (Gibco) and centrifuged to prepare for cell genome extraction.

The base editing plasmid was ABE8e++, with ABE8e as a control; and the base editing plasmid was BE4max++, with BE4max as a control.

The nSaCas9-sgRNA plasmid comprised Sa site 1 (R-loop1) (GTGGTAGACAGCATGTGTCCTA, SEQ ID NO. 17), Sa site 3 (R-loop3) (GTGTCAGGTAATGTGCTAAACA, SEQ ID NO. 18), Sa site 4 (R-loop4) (GGTGGAGGAGGGTGCATGGGGT, SEQ ID NO. 19), Sa site 5 (R-loop5) (TCTGCTTCTCCAGCCCTGGC, SEQ ID NO. 20), Sa site 6 (R-loop6) (GATGTTCCAATCAGTACGCA, SEQ ID NO. 21) and Sa site 2 (R-loop2) (ATTTACAGCCTGGCCTTTGGGG, SEQ ID NO. 32).

The sgRNA plasmid was ABLIM3-Pepper RNA described above.

### 1.3 Genome Extraction and Preparation of Amplicon Library

At 72 h after transfection, cell genome DNA was extracted with a QuickExtract™ MDNA extraction kit (QE09050) from BIOSEARCH. According to the operation procedure of a Hitom kit, corresponding identification primers (Table 2) were designed, i.e., a bridge sequence 5′-ggagtgagtacggtgtgc-3′ (SEQ ID NO. 22) was added at the 5′-terminus of a forward identification primer, and a bridge sequence 5′-gagttggatgctggatgg-3′ (SEQ ID NO. 23) was added at the 5′-terminus of a reverse identification primer, so as to obtain a first-round PCR product, and the obtained product was subjected to secondary PCR using a primer containing different barcode sequences. Subsequently, the PCR products with different labels were pooled and then underwent deep sequencing on an Illumina HiSeq platform.

TABLE 2
Target-related sequences used

| | | SEQ |
|---|---|---|
| | | ID |
| | | NO. |
| | Oligo | |
| Target | UP: CACCGTCATCCAGTGCTACCGCTG | 24 |
| name: | DN: AAACCAGCGGTAGCACTGGATGAC | 25 |
| ABLIM3 | Primer sequence (5′-3′) | |
| | F: GGAGTGAGTACGGTGTGCGTAAAGA | 26 |
| | ATCAGGGCCTCTGGATAG | |
| | R: GAGTTGGATGCTGGATGGCCTACCT | 27 |
| | TGACAGGTGAAGCAT | |

### 1.4 Data Analysis

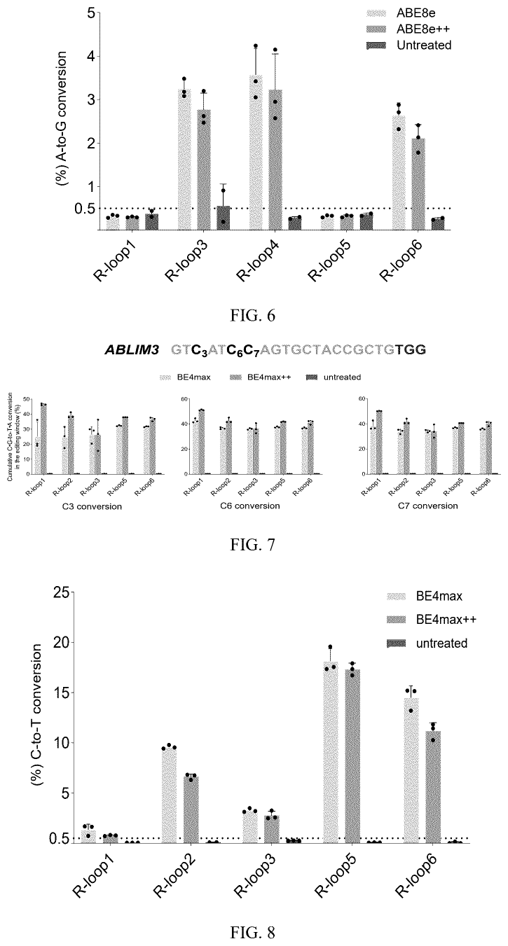

The NGS results were processed using www.rgenome.net. According to the tool's features, the analysis type of spCas9 NGG, ABE or CBE was selected for the adenine base editor. The A>G or C>T on the PAM sequence (usually 2-14 nt) was counted. The data were summarized and diagramed using Diagrampad. The editing efficiency counting results of ABE8e++ at the human endogenous target ABLIM3 are as shown in FIG. 5, and the off-target efficiency counting results of ABE8e and ABE8e++ are as shown in FIG. 6, wherein the blank control group was untreated, indicating the mutation inside the genome itself. The base editing plasmid was BE4max++, the editing efficiency counting results at the human endogenous target ABLIM3 are as shown in FIG. 7, and the off-target efficiency counting results of BE4max++ and BE4max are as shown in FIG. 8, wherein the blank control group was untreated, indicating the mutation inside the genome itself.

### Conclusion:

As shown in FIG. 5, ABE8e++ and ABE8e both can perform editing at A4 and A8 in the human endogenous target ABLIM3, but have different editing efficiencies. At the position A4, in different R-loop groups, the editing efficiencies of ABE8e++were 1.06, 1.19, 1.03, 1.08 and were 1.13 times as the original editing efficiency respectively, wherein the highest editing efficiency was 50.7% (R-loop3 group), which was improved by 8% (1.19 folds) compared with the 42.7% efficiency of the ABE8e group. At the position A8, the editing efficiencies of ABE8e++were 1.05, 1.19, 1.08, 1.08 and 1.13 times as the original editing efficiency respectively, wherein the highest editing efficiency was 50.7% (R-loop3 group), which was improved by 8.1% (1.19 folds) compared with the 42.6% efficiency of the ABE8e group. Therefore, the strategy of introducing the tDeg fusion protein into ABE can effectively improve the targeted editing efficiency.

As shown in FIG. 6, at the non-Cas9-dependent off-target level, the non-targeted editing induced by ABE8e++ was significantly reduced, with an average decrease of 0.15 fold. On R-loop3,4 and 6, ABE8e++reduced the off-target levels by 0.15, 0.10 and 0.20 fold respectively, wherein at the R-loop6 site, ABE8e++reduced the off-target levels by 0.53% (0.20 fold) (2.64% for ABE8e groups and 2.11% for ABE8e++group). Therefore, it can be seen from the above results that the strategy of introducing the tDeg fusion protein into ABE can effectively reduce the unpredictable DNA off-targeting.

As shown in FIG. 7, the tDeg system was introduced into BE4max. It can be seen from the results that the efficiencies of the fusion protein BE4max++ at different positions in the ABLIM3 target are all improved compared with those of BE4max that does not contain the protein degradation module. At the position C3, in different R-loop groups, the efficiencies of BE4max++were 1.85, 1.58, 1.19, 1.18 and 1.15 times as those of BE4max. At the position C6, in different R-loop groups, the efficiencies of BE4max++were 1.20, 1.17, 1.01, 1.16 and 1.24 times as those of BE4max. Similarly, at the position C7, in different R-loop groups, the efficiencies of BE4max++were 1.3, 1.2, 1.07, 1.1 and 1.12 times as those of BE4max. Accordingly, the strategy of introducing the tDeg fusion protein into CBE can effectively improve the targeted editing efficiency.

As shown in FIG. 8, at the non-Cas9-dependent off-target level, the off-target levels of the fusion protein BE4max++ on R-loop1, 2, 3, 5 and 6 were significantly reduced by 0.42, 0.31, 0.16, 0.04 and 0.23 fold respectively, compared with BE4max that does not contain the protein degradation module, proving that the strategy of introducing the tDeg fusion protein into CBE can also effectively reduce the unpredictable DNA off-targeting of the tDeg fusion protein. Therefore, the present invention extends to CBE, and future base editors fused with CRISPR/Cas9, such as gGBE and tBE, will have similar functions.

The above descriptions are only embodiments of the present application, but are not used for limiting the present application. For those skilled in the art, various variations and changes can be made to the present application. Any amendments, equivalent replacements, improvements and the like made within the spirit and principle of the present application should be contained within the scope of the claims of the present application.

What is claimed is:

1. A fusion protein, the fusion protein comprising a protein degradation module and a base editing module, wherein the protein degradation module comprises a degron and an RNA-binding polypeptide.

2. The fusion protein according to claim 1, wherein the protein degradation module is located at the C-terminus of the fusion protein.

3. The fusion protein according to claim 1, wherein the degron comprises an amino acid sequence as shown in SEQ ID NO. 1 or an amino acid sequence having at least 98% sequence identity to SEQ ID NO. 1; and/or, the RNA-binding polypeptide comprises an amino acid sequence as shown in SEQ ID NO. 3 or an amino acid sequence having at least 98% sequence identity to SEQ ID NO. 3.

4. The fusion protein according to claim 1, wherein the base editing module comprises glycosylase, nucleoside deaminase and/or nuclease.

5. A biological material, the biological material comprising any one of the following A)-D):
A) a gene encoding the fusion protein according to claim 1;
B) an expression cassette containing the gene in A);
C) a recombinant vector containing the gene in A) and/or the expression cassette in B); and
D) a recombinant cell or recombinant bacterium containing the fusion protein according to claim 1, the gene in A), the expression cassette in B) and/or the recombinant vector in C).

6. A base editing system, the base editing system comprising the fusion protein according to claim 1.

7. The base editing system according to claim 6, wherein the system further comprises sgRNA, the sgRNA guides the fusion protein to perform base editing on a target gene; the sgRNA further comprises an RNA-binding polypeptide response sequence which binds to the RNA-binding polypeptide.

8. Use of a protein degradation module in a base editing system, wherein the protein degradation module comprises a degron and an RNA-binding polypeptide; the use comprises reducing the off-target level of the base editing system and/or improving the gene editing efficiency of the base editing system.

9. Use of the fusion protein according to claim 1 in reducing the off-target level and/or improving the gene editing efficiency.

10. Use of the fusion protein according to claim 1 in gene editing for non-disease diagnosis and therapy purposes and/or preparation of gene editing products.

11. The fusion protein according to claim 2, the RNA-binding polypeptide and the degron are connected in sequence from the N-terminus to the C-terminus of a sequence.

12. The fusion protein according to claim 4, wherein the nucleoside deaminase comprises cytosine deaminase and/or adenosine deaminase.

13. The fusion protein according to claim 12, wherein the adenosine deaminase is TadA-8e.

14. The fusion protein according to claim 12, wherein the cytosine deaminase is rAPOBEC.

15. The fusion protein according to claim 4, wherein the nuclease is selected from one or more of Cas9, Cas3, Cas8a, Cas8b, Cas10d, Cse1, Csy1, Csn2, Cas4, Cas10, Csm2, Cmr5, Fok1 and Cpf1.

16. A base editing system, the base editing system comprising the biological material according to claim 5.

17. Use of the biological material according to claim 5 in reducing the off-target level and/or improving the gene editing efficiency.

18. Use of the base editing system according to claim 6 in reducing the off-target level and/or improving the gene editing efficiency.

19. Use of the biological material according to claim 5 in gene editing for non-disease diagnosis and therapy purposes and/or preparation of gene editing products.

20. Use of the base editing system according to claim 6 in gene editing for non-disease diagnosis and therapy purposes and/or preparation of gene editing products.
